(19) United States
(12) Patent Application Publication
(10) Pub. No.: US 2011/0124097 A1
(43) Pub. Date: May 26, 2011

(54) APPARATUS FOR REDUCING CARBON DIOXIDE CONTAINED IN COMBUSTION SMOKES

(75) Inventor: Paolo Amadesi, Bologna (IT)

(73) Assignee: LEON ENGINEERING S.P.A.

(21) Appl. No.: 13/003,551

(22) PCT Filed: Jul. 3, 2009
(86) PCT No.: PCT/IT09/00294

§ 371 Date: Jan. 10, 2011

(30) Foreign Application Priority Data
Jul. 8, 2008 (IT) BO2008A000429

(51) Int. Cl. C12M 3/00

(52) U.S. Cl. 435/292.1; 435/289.1

(57) ABSTRACT

The present invention relates to an apparatus (1) for reducing the carbon dioxide contained in combustion smokes which comprises at least one smoke inlet conduit (2) inside at least one operating chamber (3) and at least one ejection conduit (4) for the gases treated. The at least one chamber (3) comprises at least one plant (10) arranged along the smoke path between the inlet conduit (2) and the ejection conduit (4). The smokes strike the plant (10) surfaces during their circulation.

### TECHNICAL FIELD

The present invention relates to an apparatus for reducing the carbon dioxide contained in combustion smokes, in particular suitable for combustion smokes of organic substances and therefore also suitable to be used downstream of incinerators, waste to energy apparatus and other combustion apparatus.

A waste to energy apparatus actually is a waste incinerator capable of exploiting the calorific contents of the waste itself for generating heat, heating water (or other fluids) and finally producing electric energy or conveying the heated water towards rooms and areas to be warmed. Therefore, it differs from the old incinerators that only thermally destroyed waste without producing energy. The use of waste to energy apparatus looks like a solution to the problem of dumps that have become overfilled.

Incinerators are apparatus basically used for waste disposal by a high temperature combustion process (incineration) that as final products gives a gaseous effluent, ashes and dusts. Each of these apparatuses determines an emission of smokes to the atmosphere (burnt gases, a small percentage of volatile and/or suspended unburnt products, carbon dioxide and other components in a small percentage): actually, such emission constitutes the main problem of waste to energy apparatus and incinerators.

Atmospheric pollution that can be ascribed to such emissions in fact is a problem difficult to overcome.

In particular, several filtering units exist, suitable for removing the slag (volatile and/or suspended unburnt products) but an immediate reduction of the level of carbon dioxide (CO₂) is not possible.

### DISCLOSURE OF THE INVENTION

The main purpose of the present invention is to provide an apparatus for reducing the carbon dioxide contained in combustion smokes.

Within the scope of such technical purpose, another object of the present invention is to provide an apparatus for reducing the carbon dioxide contained in combustion smokes which is easy to manage and maintain.

Another object of the present invention is to provide an apparatus for reducing the carbon dioxide contained in combustion smokes suitable for favouring a consequent quick development and growth of plants useful for commercial or industrial/agricultural/food purposes.

A further object of the present invention is to provide an apparatus for reducing the carbon dioxide contained in combustion smokes of limited cost, relatively simple practical embodiment and safe application.

This purpose and this object are achieved by the present apparatus for reducing the carbon dioxide contained in combustion smokes, of the type comprising at least one smoke inlet conduit inside at least one operating chamber and at least one ejection conduit for the gases treated, characterised in that said at least one chamber comprises at least one plant arranged along the smoke path from the inlet conduit to the ejection conduit, said smokes striking the surfaces of said plant during their circulation.

### BRIEF DESCRIPTION OF THE DRAWINGS

Further details will appear more clearly from the detailed description of a preferred but non-exclusive embodiment of an apparatus for reducing the carbon dioxide contained in combustion smokes, illustrated by way of a non-limiting example in the annexed drawings, wherein:

FIG. 1 is a schematic top view of an apparatus for reducing the carbon dioxide contained in combustion smokes according to the invention;

FIG. 2 is a perspective view of a particular of apparatus.

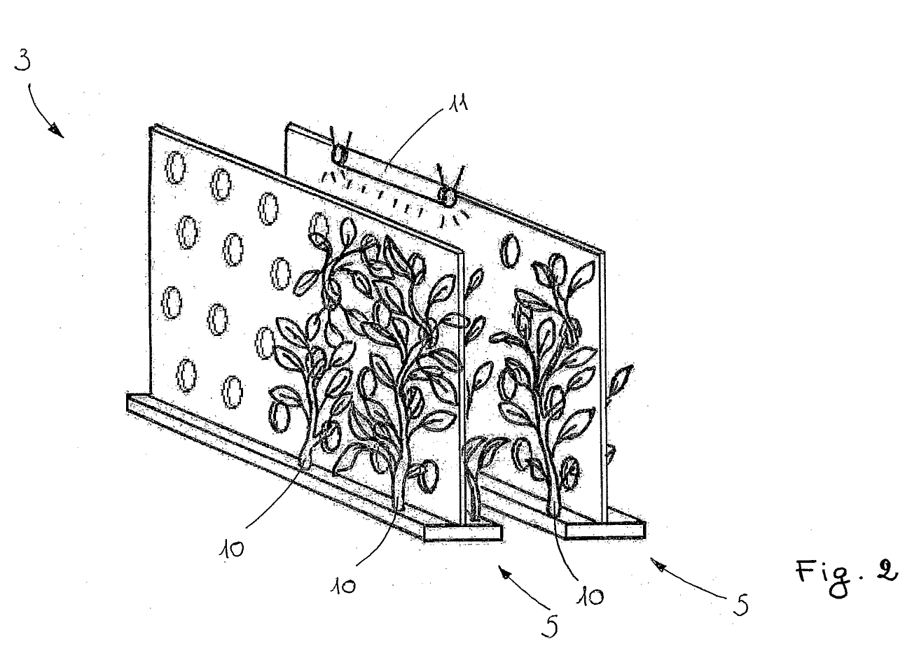

### DETAILED DESCRIPTION OF THE PREFERRED EMBODIMENTS OF THE INVENTION

With particular reference to such figures, numeral 1 globally denotes an apparatus for reducing the carbon dioxide contained in combustion smokes.

The apparatus 1 comprises at least one smoke inlet conduit 2 inside at least one operating chamber 3 and at least one ejection conduit 4 for the gases treated.

The at least one chamber 3 comprises at least one plant 10 arranged along the smoke path from the inlet conduit 2 to the ejection conduit 4.

The plant 10 is arranged in such a manner that the smokes, flowing along chamber 3 itself, strike the surfaces of plant 10 during their circulation.

The high contents of CO₂ of the smokes are a factor that predisposes to a particular efficiency and rapidity of the chlorophyllian photosynthesis of plant 10.

Chlorophyllian photosynthesis is the set of reactions during which green plants produce organic substances starting from CO₂ and from water, in the presence of light. Through chlorophyll, solar energy (light) is transformed into a form of chemical energy usable by vegetal organisms for their subsistence. Such organisms are called autotrophs.

The organic product of oxygenic photosynthesis is glucose (C₆H₁₂O₆), the most widespread monosaccharide carbohydrate. Afterwards, from this, various other macromolecules are assembled, such as starch (the build-up of carbon in plants) and sucrose (the main carrier of carbon in plants). Carbon and hydrogen to be converted into organic substance are respectively provided by carbon dioxide (CO₂) from the atmosphere and by water (H₂O). Almost all of the oxygenic photosynthesis is carried out by plants and algae that obtain hydrogen from water (H₂O). In this case, the chemical reaction that summarises the process is:

6 CO₂+6 H₂O+686 Kilocalories/moles→C₆H₁₂O₆+6 O₂

By way of an in-depth analysis, it may be said that for 1 absorbed kg of CO₂, each leaf uses 0.409 Kg of water, gives out 0.727 Kg of O₂ and its starchy body increases by 0.682 kg.

The industrial processes that produce CO₂ are combustions of two different types:
a) in the lack of nitrogen, where smokes consist almost totally of CO₂;
b) in the atmosphere, where the concentration of CO₂ is around 10/15%, with higher volume of smokes than in the previous case.

While apparatus 1 according to the invention is suitable for being associated with any “burner”, it is particularly suitable for the combustions defined at item a) (hereinafter referred to as type a) combustions). The at least one plant 10 is of the type with superficial leaf growth: in fact, it is essential that each plant 10 arranged into room 3 bases its life, growth and development activities on chlorophyllian photosynthesis. The at least one plant 10 has its roots in an inert substrate and is subject to irrigation with a nutritive solution consisting of water and of compounds required for bringing the necessary elements normally taken with mineral nutrition according to the technique called hydroponic cultivation. Such technique is known by the name of hydroculture. According to an embodiment solution of particular practical and application interest, there is a plurality of plants 10, reciprocally side by side along a line 5 aligned with the smoke path, entirely occupying the respective operating chamber 3. Suitably, in order to increase the efficiency of reduction of carbon dioxide in smokes, there is a plurality of lines 5, parallel with one another, entirely occupying the respective operating chamber 3. Always pursuing the object of minimising residual carbon dioxide in smokes after they have fully crossed apparatus 1, it is suitable to make apparatus 1 wherein there is a plurality of operating chambers 3, reciprocally arranged in a series so that the smoke ejection conduit 4 of a first chamber 3 coincides with the inlet conduit 2 of the following one. Consecutive chambers 3 are reciprocally arranged like a labyrinth of subsequent corridors 6 housing respective plants 10. Such labyrinth defines a forced route, interfering with plants 10 for the smoke flow with striking of plants 10. The fact that smokes (rich in carbon dioxide) strike plants 10 makes them take very easily all the carbon dioxide required for the photosynthesis process, releasing oxygen molecules. The efficacy of chlorophyllian photosynthesis in the presence of light is defined by quantity R defined as the absorption coefficient of CO₂ expressed in (kg of absorbed CO₂)/(h per m² of leaf surface) where h is the exposure time expressed in hours.

R=kg of abs.CO₂/m² of leaf surface.h

Such quantity R actually represents an absorption coefficient of CO₂ and directly depends on the lighting intensity I and on concentration C of carbon dioxide present in the smokes. In general:

$$\frac{\partial R}{\partial I} > 0 \frac{\partial R}{\partial C} > 0$$

Both derivatives are practically reduced to zero for limit values of I and C, that is, for I=Iasint and for C=Casint, the absorption coefficient R, beyond these limit values, reaching a maximum value defined as Rasint. The number of consecutive chambers 3 defines a corridor 6 of width B and height H imposed by construction requirements, and overall length L that may be determined through the following formula:

$$L = F \frac{Qm}{200 SHR asin t} ( m )$$

where
Rasint is the maximum absorption coefficient of CO₂ expressed in (kg of absorbed CO₂)/(h per m² of leaf surface),
H, B, L respectively are height, width and length of said corridor 6 expressed in metres,
S is the specific leaf surface expressed in (m² of leaves)/(m² of side face of corridor 6),
Qm is the mass capacity of CO₂ expressed in kg by the hour,
Qv is the volume capacity of CO₂ in m³ by the hour,
F is the reduction coefficient of CO₂.

The typical reduction coefficient F of an apparatus 1 according to the invention is in the order of 90%. The at least one chamber 3 comprises at least one light source 11 for the lighting of the respective at least one plant 10, such lighting will be suitable for favouring the photosynthesis process. Positively, such at least one light source 11 may be of the cold light type and substantially shaped as an elongated tube for the even distribution of light. It is also suitable to note that apparatus 1 may comprise suitable valve groups 7 and 8 intercepting the inlet conduit 2 and said ejection conduit 4 for inverting the smoke flow and consequently exchanging the function of said two conduits 2 and 4.

The possibility of inverting the smoke flow in the apparatus 1 determines the advantage of first impinging plants 10 located at an entrance with smokes particularly rich in carbon dioxide, subjecting them to a particularly intense activity (related to chlorophyllian photosynthesis), and then at the inversion, those located at the outlet (and therefore that so far had been struck by smokes with a reduced content of carbon dioxide). This exchange favours the ideal exploitation of plants 10 and thus ensures the achievement of maximum efficiency of apparatus 1 itself.

The apparatus 1 according to the invention may positively consist of two identical overlapped labyrinths (in turn consisting of the sequence of chambers 3), in order to alternate for each of them the lighted step to the dark step, for allowing the plant to metabolize the starch (deriving from glucose C₆H₁₂O₆) formed. By way of an example, below is the detailed description of a possible embodiment of an apparatus 1 according to the invention.

The apparatus 1 the following description refers to is that applicable to a type a) heater, with treatment capacity of about 3 ton/h, and carbon dioxide output flow rate Q_{M}=3200 kg/h (Qv˜1600 m³/h).

The prototype study has determined the value SHRₐₛᵢₙₜ=0.6.

Setting an absorption equal to 90% of carbon dioxide (2880 kg/h), we have L=2640 in, obtained with 72 chambers 3, each 0.5 m large and 38.4 m long.

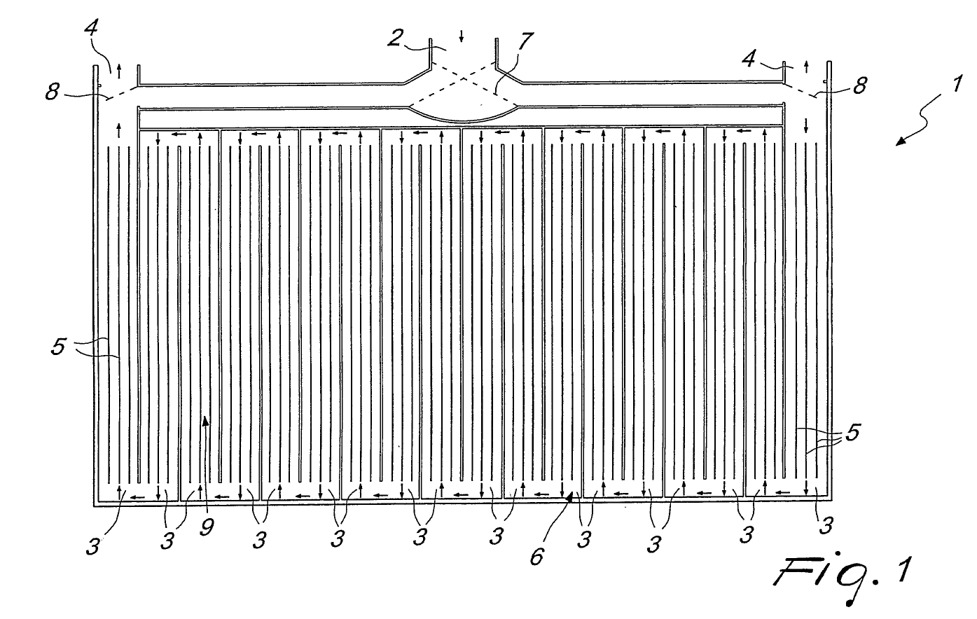

The apparatus 1 therefore has a square surface with a 38 m side and 5 m height. For simplicity, the gaseous flow is sent in n° 4 passages (delimited between the side walls of each chamber 3 and the parallel lines 5 comprised therein) forming a base group 9: in this example, apparatus 1 consists of n. 18 groups 9 in series with each other, as shown in FIG. 1.

Lines S consist of suitable panels on both surfaces whereof climbing plants 10 with multiple leaf development are hydroponically grown. The panels are supported by a suitable metal structure with section bars, forming side by side portals each 38.4 metre long, and supported every 6.4 metres by pillars of metal section bars 5 in high. The portals are connected to one another by cross section bars bolted at the top and at the bottom. Each portal 12 supports panels 3.2 m large and 5 m high side by side, consisting of composite material for example 30 mm thick, provided on both faces with small holes far from one another for example by 100 mm, designed for constituting an optimum anchoring surface for the climbing plants. Each panel is provided at the bottom part thereof with a suitable duct containing the hydroponic support material for the roots and suitable for being hydrically impregnated drop by drop through a vertical conduit of plastic material located at an end of the panel and fed by a conduit located at the top.

The panels of each portal are laterally connected to one another by multiple hinge metal couplings, whereas the edges are coated with semi-cylindrical rubber seals or the like that ensure the interstitial gas seal. Periodical maintenance is preceded by the extraction of one or more rows of panels from the top from the corresponding portal by a bridge crane about 12 m high.

N° 36 identical portals are side by side at a reciprocal distance of 0.5 m. The portals are connected to each other by cross section bars bolted at the top and at the bottom.

The interval between each portal at each side by side pair of panel lines is covered by a series of “roofs” of a material similar to that of the panels, each sized 3.2×1.0 metres liftable by said bridge crane. A dual vertical cold light lighting tube is stiffly hung to each “roof”, each tube has an electrical power of 50 Watts, for a total of 1824 single tubes and about 92 electrical kW used. Through a conduit system and n. 3 gas deviation valves (valve groups 7 and 8) it is possible to invert the gas flow itself, so as to periodically replace the more used initial leaf zone with the final one. It is therefore suitable for the number of groups 9 in a series to be even, so that both the inlet conduit 2 and the ejection conduit 4 are located on the same side of apparatus 1.

It is suitable to note that apparatus 1 according to the invention may ensure very high reductions of the level of carbon dioxide contained in smokes and it allows an easy operation of apparatus 1 as well as optimum maintenance of the components thereof. Such apparatus 1, however, may also be used for different purposes, for example using the quick growth of the plants used in apparatus 1.

The quick growth (ensured by the optimum environmental conditions the plants are in) allows obtaining plants with interesting commercial sizes in a short time (compared to a standard cultivation in greenhouse at atmospheric conditions). It is therefore possible to use apparatus 1 according to the invention for combining the effects of reduction of carbon dioxide into the discharge smokes with a cultivation (for sales purpose) of plants of various commercial interest. In fact, it is possible to consider cultivating decorative plants, for feeding purpose (either human or animal). The growth acceleration of the plants into chambers 3 in fact allows quickly bringing them from very small dimensions to commercial dimensions. It has thus been seen that the invention achieves the intended objects. Several changes and variations can be made to the invention thus conceived, all falling within the scope of the inventive concept. Moreover, all details can be replaced with other technically equivalent ones. In the illustrated examples of embodiments, single features described with reference to specific examples may actually be interchanged with other different features, existing in other examples of embodiments. Moreover, it should be noted that should any things be found to be already known during the patent issue procedure, they should be understood as not claimed and disclaimed from the claims.

The embodiment of the present invention will be carried out with the utmost observance of law and regulatory provisions of the products object of the invention or correlated thereto and with the authorisation, if required, of the relevant competent authorities with particular reference to safety, enviromnental pollution and health related standards.

In the practice, the materials used as well as the shapes and sizes may be whatever, according to the requirements, without departing from the scope of protection of the following claims.

1. An apparatus for reducing the carbon dioxide contained in combustion smokes, of the type comprising at least one smoke inlet conduit (2) inside at least one operating chamber (3) and at least one ejection conduit (4) for the gases treated, characterised in that said at least one chamber (3) comprises at least one plant (10) arranged along the smoke path between the inlet conduit (2) and the ejection conduit (4), said smokes striking the surfaces of said plant (10) during their circulation.

2. The apparatus according to claim 1, characterised in that said at least one plant (10) is of the type with superficial leaf growth, said at least one plant (10) basing its life, growth and development activities on chlorophyllian photosynthesis.

3. The apparatus according to claim 1, characterised in that said at least one plant (10) has its roots in an inert substrate and is subject to irrigation with a nutritive solution consisting of water and of compounds required for bringing the necessary elements normally taken with mineral nutrition by the plant in nature, according to the technique called hydroponic cultivation.

4. The apparatus according to claim 1, characterised in that it comprises a plurality of said plants (10), reciprocally side by side along a line (5) aligned with the smoke path entirely occupying the respective operating chamber (3).

5. The apparatus according to claim 4, characterised in that it comprises a plurality of lines (5), parallel with each other, entirely occupying the respective operating chamber (3).

6. The apparatus according to claim 1, characterised in that there is a plurality of said operating chambers (3), reciprocally arranged in a series so that the smoke ejection conduit (4) of a first chamber (3) coincides with the inlet conduit (2) of the following one.

7. The apparatus according to claim 6, characterised in that said consecutive chambers (3) are reciprocally arranged as a labyrinth of subsequent corridors (6) housing respective plants (10), labyrinth defining a forced route, interfering with said plants (10), for the smoke flow with striking of the plants (10) themselves.

8. The apparatus according to claim 6, characterised in that the number of consecutive chambers (3) defines a corridor (6) of width and height imposed by construction requirements and overall length that may be determined through the following formula:
$$L = F \frac{Qm}{200 SHR asin t} ( m )$$
where Rasint is the maximum absorption coefficient of CO₂ expressed in (kg of absorbed CO₂)/(h per m² of leaf surface),
H, B, L respectively are height, width and length of said corridor (6) expressed in metres,
S is the specific leaf surface expressed in (m² of leaves)/(m² of side face of corridor (6)),
Qm is the mass capacity of CO₂ expressed in kg by the hour,
Qv is the volume capacity of CO₂ in m³ by the hour,
F is the reduction coefficient of CO₂.

9. The apparatus according to claim 1, characterised in that said at least one chamber (3) comprises at least one light source (11) for the lighting of the respective at least one plant (10), lighting suitable for favouring the photosynthesis process.

10. The apparatus according to claim 9, characterised in that said at least one light source (11) is of the cold light type substantially shaped as an elongated tube for the even distribution of light.

11. The apparatus according to one or more of the previous claims, characterised in that it comprises suitable valve groups (7, 8) intercepting said at least one inlet conduit (2) and said at least one ejection conduit (4) for inverting the smoke flow and consequently exchanging the function of said two conduits (8, 7).
